(19) United States
(12) Patent Application Publication
(10) Pub. No.: US 2025/0352492 A1
(43) Pub. Date: Nov. 20, 2025

(54) MODULAR TOPICAL PATCH SYSTEM FOR PAIN RELIEF

(71) Applicant: Kenvue Brands LLC, Summit, NJ (US)

(72) Inventors: Max Hunter KNECHT, Brooklyn, NY (US); Andrew GLOWACKI, Hatfield, PA (US)

(21) Appl. No.: 19/210,054

(22) Filed: May 16, 2025

Related U.S. Application Data
(60) Provisional application No. 63/648,447, filed on May 16, 2024.

(51) Int. Cl. A61K 9/70

(52) U.S. Cl. CPC A61K 9/703

(57) ABSTRACT

A modular topical patch system may include two or more topical patches that share a commonality to allow them to be affixed adjacent, parallel or in close proximity to one another on skin of a user to facilitate pain relief. The commonality may be a common dimension, a common angle, a complementary angle, a common shape and/or a complementary shape

### PRIORITY CLAIM

The present application claims priority to U.S. Provisional Application Ser. No. 63/648,447 filed May 16, 2024, the entire contents of which is hereby incorporated by reference herein.

### FIELD OF THE INVENTION

The present invention relates to a modular topical patch system that includes two or more patches that share a commonality to allow them to be affixed adjacent, parallel or in close proximity to one another on the skin to facilitate pain relief. The commonality may be a common dimension, a common angle, a complementary angle, a common shape and/or a complementary shape. Additionally, the present invention relates to methods of relieving pain, where a user affixes two or more topical patches adjacent, parallel or in close proximity to one another on the skin to facilitate pain relief, where the two or more topical patches share a commonality that facilitates modular application to the skin.

### BACKGROUND OF THE INVENTION

Pain is a common experience that may be of short duration (called “acute pain”) or of longer duration, for example, six months or longer (called “chronic pain”). Pain may negatively impact a person's quality of life and treatment of pain may be costly. Pain treatment options that are effective and inexpensive are desirable.

Body pain is often treated with topical creams and patches. However, it is estimated that one in four consumers are not satisfied with their topical product experience. Regarding topical patches, some consumers note that commercially available patches are uncomfortable to wear and interfere with their daily activities and exercise. Other consumers complain that commercially available topical patches peel or fall off during use, or that they are uncomfortable or embarrassing to wear.

Some consumers find that commercially available topical patches are not the right size, material and/or shape to treat all portions of their body in pain. For example, the commercially available topical patches may be too large or too inflexible to fit around smaller joints like the ankle, fingers, knee, wrist or elbow. For example, some commercially available topical patches are in the shape of a large rectangle. These patches, while perhaps suitable for large areas like the back, are not suitable for most other parts of the body.

As such, there exists a need for improvements in topical patches and in the use of topical patches to provide superior pain relief to consumers. Such improvements shall take into account different parts of the body that may be affected by pain and adaptability of the topical patch or topical patch system to user preference.

### BRIEF DESCRIPTION OF THE DRAWINGS

FIG. 1A is a top plan view of two hexagonal topical patches packaged for easy removal and modular use by a consumer.

FIG. 1B is a side view of the two hexagonal topical patches packaged for easy removal and modular use by a consumer of FIG. 1A.

FIG. 1C is a front view of the two hexagonal topical patches packaged for easy removal and modular use by a consumer of FIG. 1A.

FIG. 1D is a top perspective view of two hexagonal topical patches packaged for easy removal and modular use by a consumer.

FIG. 1E is a bottom perspective view of two hexagonal topical patches packaged for easy removal and modular use by a consumer.

FIG. 2A is a top plan view of two hexagonal topical patches packaged for easy removal and modular use by a consumer.

FIG. 2B is a side view of the two hexagonal topical patches packaged for easy removal and modular use by a consumer of FIG. 2A.

FIG. 2C is a front view of the two hexagonal topical patches packaged for easy removal and modular use by a consumer of FIG. 2A.

FIG. 2D is a top perspective view of two hexagonal topical patches packaged for easy removal and modular use by a consumer.

FIG. 2E is a bottom perspective view of two hexagonal topical patches packaged for easy removal and modular use by a consumer.

FIG. 3A is a top plan view of a hexagonal topical patch packaged for easy removal and modular use by a consumer.

FIG. 3B is a side view of a hexagonal topical patch packaged for easy removal and modular use by a consumer of FIG. 3A.

FIG. 3C is a front view of a hexagonal topical patch packaged for easy removal and modular use by a consumer of FIG. 3A.

FIG. 3D is a top perspective view of a hexagonal topical patch packaged for easy removal and modular use by a consumer.

FIG. 3E is a bottom perspective view of a hexagonal topical patch packaged for easy removal and modular use by a consumer.

FIG. 4A is a top plan view of two triangular topical patches packaged for easy removal and modular use by a consumer.

FIG. 4B is a side view of two triangular topical patches packaged for easy removal and modular use by a consumer of FIG. 4A.

FIG. 4C is a front view of two triangular topical patches packaged for easy removal and modular use by a consumer of FIG. 4A.

FIG. 4D is a top perspective view of two triangular topical patches packaged for easy removal and modular use by a consumer.

FIG. 4E is a bottom perspective view of two triangular topical patches packaged for easy removal and modular use by a consumer.

FIG. 5A is a top plan view of two windshield-shaped topical patches packaged for easy removal and modular use by a consumer.

FIG. 5B is a side view of the two windshield-shaped topical patches packaged for easy removal and modular use by a consumer of FIG. 5A.

FIG. 5C is a front view of the two windshield-shaped topical patches packaged for easy removal and modular use by a consumer of FIG. 5A.

FIG. 5D is a top perspective view of two windshield-shaped topical patches packaged for easy removal and modular use by a consumer.

FIG. 5E is a bottom perspective view of two windshield-shaped topical patches packaged for easy removal and modular use by a consumer.

FIG. 6A is a top plan view of a triangular topical patch packaged for easy removal and modular use by a consumer.

FIG. 6B is a side view of the triangular topical patch packaged for easy removal and modular use by a consumer of FIG. 6A.

FIG. 6C is a front view of the triangular topical patch packaged for easy removal and modular use by a consumer of FIG. 6A.

FIG. 6D is a top perspective view of a triangular topical patch packaged for easy removal and modular use by a consumer.

FIG. 6E is a bottom perspective view of a triangular topical patch packaged for easy removal and modular use by a consumer.

FIGS. 7A-7B depict unfolded views of a carton to package one or more topical patches.

FIGS. 8A-8B depict a front and rear view of a pouch to package one or more topical patches.

FIGS. 9A-9B depict different ornamental examples of the front view of a carton to package one or more topical patches.

FIGS. 10A-10B, 11A-B and 12A-B depict various examples of modular topical patch systems that may be used alone or in combination.

FIG. 13 depicts a commercially available topical patch.

FIGS. 14A-14B depict an example of a modular topical patch system using small hexagon patches.

FIGS. 15A-15B depict an example of a modular topical patch system using medium hexagon patches.

FIGS. 16A-16B depict an example of a modular topical patch system using large hexagon patches.

FIGS. 17A-17B depict an example of a modular topical patch system using small triangular patches.

FIGS. 18A-18B depict an example of a modular topical patch system using medium triangular, or windshield-shaped, patches.

FIGS. 19A-19B depict an example of a modular topical patch system using large triangular patches.

FIG. 20 depicts an example of a modular patch system using different sized and shaped topical patches.

### SUMMARY OF THE INVENTION

A modular topical patch system may include two or more topical patches that share a commonality to allow them to be affixed adjacent, parallel or in close proximity to one another on skin of a user to facilitate pain relief. The commonality may be a common dimension, a common angle, a complementary angle, a common shape and/or a complementary shape. According to one example, the common angle is an obtuse angle or an acute angle. According to another example, the complementary angle of a first patch is an obtuse angle and the complementary angle of a second patch is an acute angle. In a further example, the two or more topical patches are positioned on the skin at a smaller joint or body location of the user.

A method of relieving pain may further include a user affixing two or more topical patches adjacent, parallel or in close proximity to one another on skin of a user to facilitate pain relief, wherein the two or more topical patches share a commonality that facilitates modular application to the skin. The commonality may be a common dimension, a common angle, a complementary angle, a common shape and/or a complementary shape. According to one example, the common angle may be an obtuse angle or an acute angle. According to another example, the complementary angle of a first patch may be an obtuse angle and the complementary angle of a second patch may be an acute angle. In a further example, the method may include the two or more topical patches being positioned on the skin at a smaller joint or body location of the user.

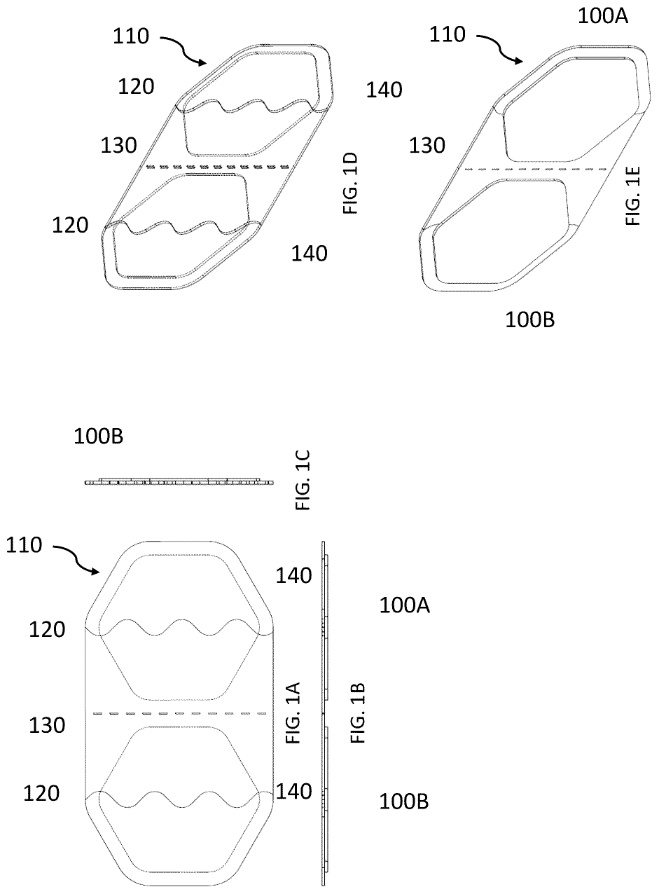

The present invention further covers the ornamental design for a modular topical patch system as shown and described in FIGS. 1A through 9B.

### DETAILED DESCRIPTION

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination. Unless otherwise defined, all technical and scientific terms used herein have the same meaning as commonly understood by one of ordinary skill in the art to which this disclosure pertains. The terminology used in the description is for describing particular examples only and is not intended to be limiting of the disclosure.

In the disclosure, the singular forms “a,” “an,” and “the” include the plural reference, and reference to a particular numerical value includes at least that particular value, unless the context clearly indicates otherwise. Thus, e.g., a reference to “a material” is a reference to at least one of such materials and equivalents thereof known to those skilled in the art, and so forth.

When a value is expressed as an approximation by use of the descriptor “about” it will be understood that the particular value forms another example. In general, use of the term “about” indicates approximations that can vary depending on the desired properties sought to be obtained by the disclosed subject matter and is to be interpreted in the specific context in which it is used, based on its function. The person skilled in the art will be able to interpret this as a matter of routine. In some cases, the number of significant figures used for a particular value may be one non-limiting method of determining the extent of the word “about.” In other cases, the gradations used in a series of values may be used to determine the intended range available to the term “about” for each value. In some cases, the descriptor “about” will be understood as meaning ±up to 10%, for example ±10, 9, 8, 7, 6, 5, 4, 3, 2 or 1%. Where present, all ranges are inclusive and combinable. That is, references to values stated in ranges include every value within that range.

When a list is presented, unless stated otherwise, it is to be understood that each individual element of that list and every combination of that list is to be interpreted as a separate example. For example, a list of examples presented as “A, B, or C” is to be interpreted as including the examples, “A,” “B,” “C,” “A or B,” “A or C,” “B or C,” or “A, B, or C.”

It is to be appreciated that certain features of the invention which are, for clarity, described herein in the context of separate examples, may also be provided in combination in a single example. That is, unless obviously incompatible or excluded, each individual example is deemed to be combinable with any other example(s) and such a combination is considered to be another example. Conversely, various features of the invention that are, for brevity, described in the context of a single example, may also be provided separately or in any sub-combination. It is further noted that the claims may be drafted to exclude an optional element. As such, this statement is intended to serve as antecedent basis for use of such exclusive terminology as “only” and the like in connection with the recitation of claim elements, or use of a “negative” limitation. Finally, while an example may be described as part of a series of steps or part of a more general structure, each said step may also be considered an independent example in itself.

It is believed that one skilled in the art can, based on the description herein, utilize the present invention to its fullest extent. The following specific examples are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Also, all publications, patent applications, patents, and other references mentioned herein are incorporated by reference.

Unless otherwise indicated, percentages used to express amounts of ingredients are percentage by weight (referred to as “weight %,” “wt %”, “% wt,” “% by weight” or “% (W/W)”). Similarly, weight ratios used to express relative proportions of ingredients are also determined using percentage by weight (i.e., weight ratios are calculated by dividing the percentage by weight of one ingredient by another). Unless stated otherwise, all ranges are inclusive of the endpoints, e.g., “from 4 to 9” or “from about 4 to about 9” includes the endpoints 4 and 9.

As used herein, a “product” is optionally in finished packaged form. In one example, the package includes a backing material and/or a release liner, and/or the package is a container such as a pouch or wrapper containing the composition. In one example, the composition may be applied to the skin using an applicator sheet, backing material, release liner or other functional applicator. The product may further contain additional packaging such as a plastic or cardboard box for storing such container. In one example, the product includes a composition of the invention and contains instructions directing the user to apply the composition to the skin.

As used herein, “topically applying” means directly laying on or applying to the outer skin, by use of the hands or an applicator sheet or other functional applicator.

As used herein, “pain relief” refers to the alleviation of pain at one or more locations of the body.

As used herein, “cosmetically acceptable” means that the ingredients or materials the term describes are suitable for use in contact with tissues (e.g., the skin or hair) without undue toxicity, incompatibility, instability, irritation, allergic response, or the like.

As used herein, the term “safe and effective amount” means an amount sufficient to induce the desired effect, but low enough to avoid serious side effects. The safe and effective amount of the compound, extract, or composition will vary with, e.g., the age, health and environmental exposure of the end user, the duration and nature of the treatment, the specific extract, ingredient, or composition employed, the particular carrier utilized, and like factors.

As used herein, “analgesic” means a class of drugs designed to relieve pain without causing the loss of consciousness. Such analgesics may be over-the-counter (OTC) or prescription drugs. Examples of analgesics may include, for example, acetaminophen, aspirin, COX inhibitors, and nonsteroidal anti-inflammatory drugs (NSAIDs) and salts and derivatives thereof.

A “topical analgesic” refers to a drug, medication or pain-relieving agent that is applied on the skin to relieve muscle, joint or nerve pain. Topical analgesics may also be classified as counterirritants or anti-inflammatory agents in certain cases. Topical analgesics may be absorbed by the skin and may act on the tissue beneath. Examples of suitable topical analgesics may include lidocaine, capsaicin, nonsteroidal anti-inflammatory drugs (NSAIDs), salicylate rubefacients, benzocaine, tetracaine, nitroglycerin, rubefacients, such as camphor, menthol, methyl nicotinate and methyl salicylate, and salicylates, such as aspirin, magnesium salicylate and sodium salicylate and salts and derivatives thereof. Capsaicin, menthol, methyl salicylate and camphor, for example, may also be classified as counter-irritants. A topical analgesic may include a combination of those listed above.

According to an example, the analgesic or topical analgesic may be recommended for external application to the skin, also referred to as the “exterior skin portion” of a user's body. For example, the topical analgesic should not be applied to internal body surfaces, such as the oral cavity. For this reason, the term “external analgesic” may be used simultaneously or in lieu of “topical analgesic.”

In one example, the analgesic or topical analgesic (or combination of analgesics or topical analgesics) may be present in amounts ranging from about 0.1 to about 70 wt. % of the total composition. The analgesic or topical analgesic (or combination of analgesics or topical analgesics) may be present in an amount of about 5, 10, 15, 20, 25, 30, 35, 40, 45, 50, 55, 60, 65 or 70 wt. %.

In one example, the analgesic or topical analgesic (or combination of analgesics or topical analgesics) may be present in amounts ranging from about 0.1, 0.5, 1, 1.5, 2, 2.5, 3.1, 3.2, 3.3, 3.4, 3.5, 3.6, 3.7, 3.8, 3.9, 4, 4.5, or 5 to about 3.5, 3.6, 3.7, 3.8, 3.9, 4, 4.1, 4.2, 4.3, 4.4, 4.5, 4.6, 4.7, 4.8, 4.9, 5, 5.5, 6, 6.5, 7, 7.5, 8, 8.5, 9.5, 10, 10.5, 11, 11.5, 12, 12.5, 13, 13.5, 14, 14.5, 15, 15.5, or 16 wt. % of the total composition. In one or more examples, the analgesic or topical analgesic is present in an amount ranging from about 0.1 wt. % to about 16 wt. % by total weight of the composition. In some examples, the analgesic or topical analgesic is present in an amount ranging from about 0.5 wt. % to about 8 wt. % by total weight of the composition. In one or more examples, the analgesic or topical analgesic is present in an amount ranging from about 2 wt. % to about 6 wt. % by total weight of the composition. In some examples, the analgesic or topical analgesic is present in an amount ranging from about 3 wt. % to about 5 wt. % by total weight of the composition. In one or more examples, the analgesic or topical analgesic is present in an amount ranging from about 3.5 wt. % to about 4.5 wt. % by total weight of the composition. In some examples, the analgesic or topical analgesic is present in an amount of about 4 wt. % by total weight of the composition. In some examples, the analgesic or topical analgesic is present in an amount of no more than 4 wt. % by total weight of the composition.

In one or more examples, the topical analgesic comprises lidocaine, and is present in an amount ranging from about 0.1 wt. % to about 4 wt. % by total weight of the composition. In some examples, the lidocaine is present in an amount of about 4 wt. % by total weight of the composition or in an amount of no more than 4 wt. % by total weight of the composition. Lidocaine may be present in a salt form such as lidocaine hydrochloride. Alternatively, the lidocaine may be present in a base form.

A “patch” or “topical patch” is defined as a medicated adhesive patch that is placed on the skin to deliver a specific dose of medication to the skin for local delivery of an active agent. The terms “patch” and “topical patch” may be used interchangeably herein. Alternatively, a “transdermal patch” is defined as a medicated adhesive patch that is placed on the skin to deliver a specific dose of medication through the skin and into the bloodstream. It is foreseen that references to patches or topical patches in this description could be replaced by transdermal patches in applicable examples.

For the purposes of this application, the term “modular” is defined as the application of two or more individual patches so that the patches can be easily arranged into different functional forms or spread across different body locations to facilitate more effective pain relief.

For the purposes of this application, the term “commonality” means the state of sharing features or attributes.

For the purposes of this application, the term “obtuse angle” refers to an angle that is greater than 90 degrees but less than 180 degrees. The term “acute angle” refers to an angle measuring less than 90 degrees. The term “right angle” refers to an angle measuring at about 90 degrees.

According to one example, one or more topical patches are in one or more shapes and sizes to facilitate modular application to different parts of the body. According to one example, the patch may come in a hexagonal or triangular shape. According to another example, the topical patch(es) may contain an obtuse angle or acute angle to help maintain patch structural integrity and prevent peeling. According to an example, the design of the topical patch(es) may exclude right angles.

According to another example, the patch may come in one or more sizes. According to one example, the patch may come in a small size defined as about 2 inches in length and width. According to another example, the patch may come in a medium size defined as about 3 inches in length and about 2 inches in width. According to a further example, the patch may come in a large size defined as about 4 inches in length and about 3.5 inches in width.

According to an example, the modular patch system may have several sizes of patches that are adapted to fit together to facilitate modular application to the skin for pain relief. For example, the modular patch system may include combinations of small, medium and/or large patches.

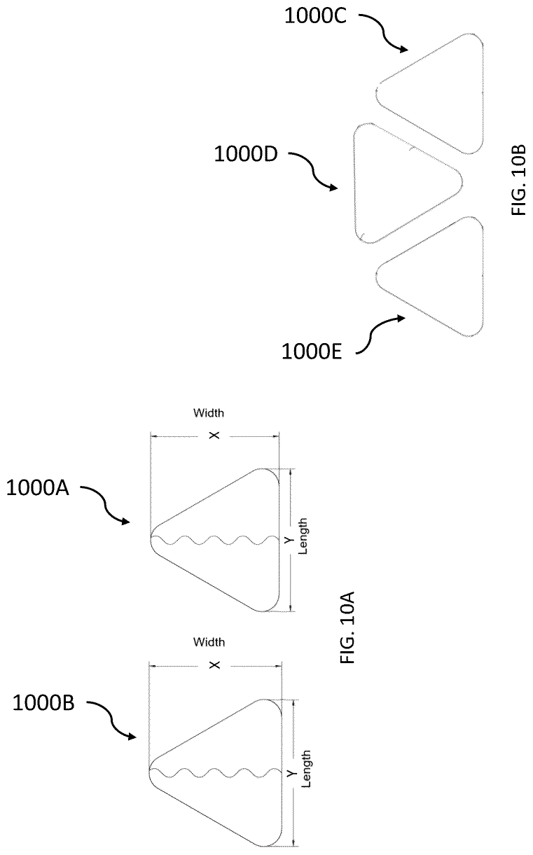

In one example, as shown in FIG. 10A, the topical patches may have a commonality, for example a common dimension, a common angle and/or a complementary shape that allows the patches to be placed adjacent to one another on the skin for modular pain relief. For example, the patches 1000A, 1000B may have a common shape and/or at least one overall side that has a common length, such as width “X” and/or length “Y.” Alternatively, the patches may have a common angle, for example an obtuse angle or an acute angle. For the purposes of this application, a “common angle” refers to two shapes having a same angle in common. The patch design may exclude a right angle to preserve structural integrity and to prevent peeling. This commonality, for example, a common shape, angle and/or dimension, may allow two or more topical patches 1000C, 1000D, 1000E to be placed adjacent, parallel or in close proximity to one another (as shown in FIG. 10B).

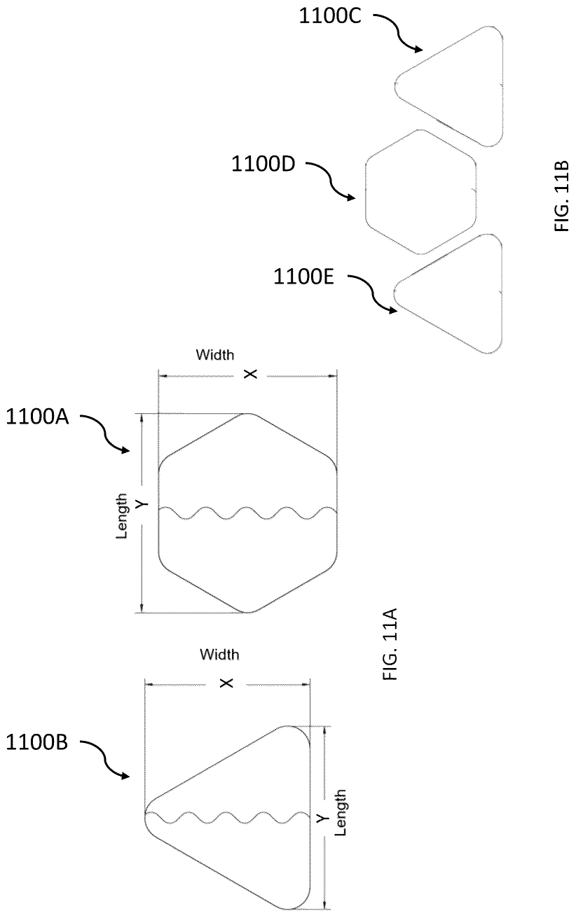

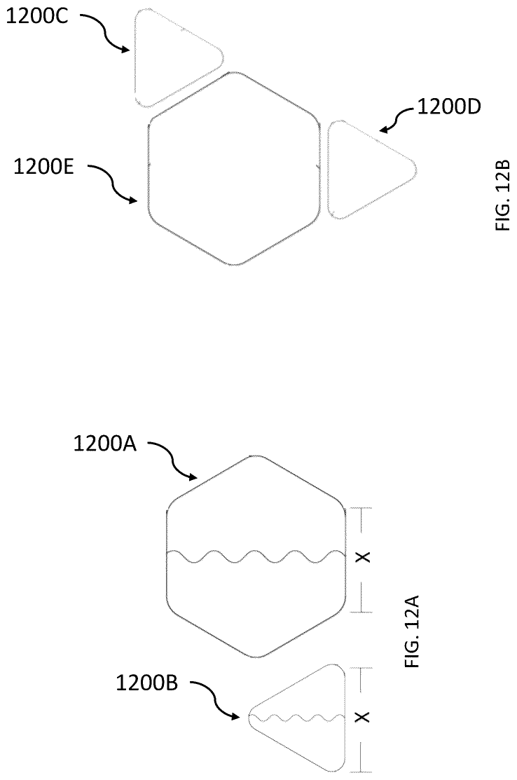

In another example, as shown in FIG. 11A, patches of two different, but complementary, shapes, e.g., a hexagonal patch 1100A and a triangular patch 1100B, may have the same overall width “X” and/or the same overall length “Y.” For the purposes of this application, the term “complementary shape” means that two or more patches are of different shapes, but the shapes allow the two or more patches to be placed adjacent, parallel or in close proximity to one another. Alternatively, the patches may have two different, but complementary angles, for example an obtuse angle or an acute angle. A complementary angle may be such that a side of one patch can be positioned parallel to a side of another patch having a different shape. In one example, one patch may have an obtuse angle and the other patch may have an acute angle that allows parallel positioning. For the purposes of this application, a “complementary angle” means that two or more patches have a different angle, but that the different angle of each patch allows the patches to be placed adjacent, parallel or in close proximity to one another. In another example, the design of both patches may exclude a right angle. As shown in FIG. 11B, a common overall width and/or length, or a complementary angle, may allow the different, but complementary, shaped topical patches 1100C, 1100D, 1100E to be placed adjacent, parallel or in close proximity to one another (as shown below).

In an alternative example, as shown in FIG. 12A, one side of a hexagonal topical patch 1200A may be the same length “X” as one side of a different, but complementary shaped, patch (triangular patch 1200B), as shown below. In one example, the different, but complementary, shaped topical patches may be triangular and hexagonal shaped.

Alternatively, the patches may have two different, but complementary angles, for example an obtuse angle or an acute angle. A complementary angle may be such that a side of one patch can be positioned parallel to a side of another patch having a different shape. In one example, one patch may have an obtuse angle and the other patch may have an acute angle that allows parallel positioning. In another example, the design of both patches may exclude a right angle. In this example, a common length “X,” or a complementary angle, may allow two or more different, but complementary, shaped topical patches to be placed adjacent, parallel or in close proximity to one another. In the example shown in FIG. 12B, two smaller triangular topical patches 1200C, 1200D are placed adjacent to a larger hexagonal topical patch 1200E.

In an example, the topical patches may be placed in close proximity to one another for modular and personalized pain relief. In close proximity may mean, for example, placing the patches about 1 mm, about 2 mm, about 3 mm, about 4 mm, about 5 mm, about 6 mm, about 7 mm, about 8 mm, about 9 mm, about 1 cm, about 1.5 cm, about 2 cm, about 2.5 cm, about 3 cm, about 3.5 cm, about 4 cm, about 4.5 cm, or about 5 cm apart. This distance of close proximity may be larger if the body part is larger. For example, patches placed on the wrist may be spaced closer together than patches placed on the shoulder. The placement of the patches may be at the discretion of the use or at the direction of the package label or a health care professional to maximize relief.

In an example, the topical patches may be adapted to provide discreet relief to users. For example, the patches may be skin-toned, assume the skin-tone of the user or may be transparent. The topical patches may feel discreet by being adapted to flex or move with the skin while body parts are in motion. The topical patches may also be sweat resistant adding to the versatility and discreetness of use, for example, while exercising or at the beach. Discreetness may also relate to the smell or scent of the topical patch. Some users may prefer a topical patch that is odorless or that has a pleasant smell, rather than a heavy therapeutic or clinical smell (for example, a strong menthol odor). In a preferred example, the user of the topical patch will not feel it during use and the topical patch will not be visible or detectable by others. This will allow the user to move with freedom and confidence.

In one example, the topical patches may be adapted to provide long-lasting pain relief. For example, such topical patches may remain affixed to the skin and release medication (e.g., lidocaine) for up to four (4) hours, up to six (6) hours, up to eight (8) hours, up to ten (10) hours, up to twelve (12) hours, up to fourteen (14) hours, up to sixteen (16) hours, up to eighteen (18) hours, up to twenty (20) hours, up to twenty-two (22) hours, or up to twenty-four (24) hours.

The topical patch may be made of material to allow flexible adhesion where the topical patch contours to a user's skin, resisting pulling or loosening no matter how much the user moves. Such advanced adhesion may maintain complete skin contact, even in high flex body locations like the joints. The flexible material in combination with the patch shape and size may provide even stronger adhesion and flexibility during use.

In an example, the use of obtuse and acute angles, instead of right angles, in the patch shape may provide structural integrity and anti-peeling benefits. Current users of commercially available topical patches often complain that patch peeling limits their activity and ability to get pain relief. Peeling may also be prevented by the use of edge-to-edge adhesive on the topical patch.

The topical patch may be easy to apply and easy to remove. The topical patch may be made of breathable material to prevent sensitivity, irritation and breakage of the skin. The topical patch should also leave no residue behind upon removal, for example tacky adhesive or the like.

In one example, the topical patch may be a hydrocolloid patch having high adhesion to skin area which necessitates pain relief. Such hydrocolloids may be designed to stay on the skin for 6 hours, or for 8 hours, or 12 hours, or 18 hours, or 24 hours, or more. The hydrocolloid patch may be formed of a polymeric film coated with a hydrocolloid material having adhesive characteristics. The polymeric film may be comprised of polymers that are synthetically manufactured. The polymeric film may be a polyurethane film. The hydrocolloid patch may also be translucent. The hydrocolloid may be a water absorbing and/or water-swellable material. The hydrocolloid may be synthetic or naturally occurring. The hydrocolloid may, for example, be crosslinked. The hydrocolloid may, for example, be selected from polyvinyl alcohol, polyvinyl acetate, polyvinylpyrolidone, polyacrylic acid, polyhydroxyalkyl acrylates, polyacrylamides, high molecular weight polyethylene glycols, polypropylene glycols, carboxymethylcellulose (CMC) (e.g. sodium carboxymethyl cellulose, cross-linked sodium carboxymethylcellulose, and/or crystalline carboxymethylcellulose), pectin, gelatin, high molecular weight carbowax, carboxypolymethylene, carboxymethyl starches, alginates, carrageenan, gelatin, citrus pectin, powdered pectin, synthetic or natural gums such as guar gum, gum Arabic, locust bean gum, tragacanth gum, dextran, starches, karaya, starch-acrylonitrile graft copolymer and mixtures thereof. Preferred hydrocolloids may be carboxymethylcellulose, alginates, and pectin. The hydrocolloid may be particles, for example having an average particle size of from about 1 μm to about 400 μm.

In another example, the topical patch may be a UV-cured hydrogel patch having high adhesion to skin area which necessitates pain relief. Such UV-cured hydrogel patches may be designed to stay on the skin for 6 hours, or for 8 hours, or 12 hours, or 18 hours, or 24 hours, or more. The UV-cured hydrogel may comprise one or more of the following ingredients: adhesives such as diacetone acrylamide-acrylamidomethylpropane sulfonic acid copolymer, penetration enhancers such as diethylene glycol monoethylether, preservatives such as ethylparaben or methylparaben, emollients such as isopropyl laureate, surfactants such as polysorbate 80, as well as other ingredients such as polyvinylpyrrolidone, methylparaben, glycerin, water, propylene glycol, and/or sodium hydroxide. The UV-cured hydrogel may further include an adhesive to attach it to a backing material and/or release liner, as described below.

The topical patch material may be applied to or embedded on or into a scrim, i.e., a strong coarse fabric or fiber network, that acts to maintain the structural integrity of the patch. The scrim may be a random network of fibers or may be patterned. In one example, the scrim is a random network of fibers which adds additional discreetness and blending with skin tone.

The topical patch may further include an adhesive surface or tacky layer for application and adherence to the skin of a user. The adhesive may be applied from edge-to-edge of the patch for stronger adhesion.

The patch of the present invention may also comprise a backing material formed of a sheet, and/or a release liner. The sheet of backing material may be comprised of woven fabric or polymeric films such as polyolefin (e.g., polyethylene) polyvinylchloride (PVC), polyester, latex or polyurethane films.

The topical patch of the present invention may comprise a release liner system. The release liner system may include a plurality of protective sheets that isolate the adhesive surfaces of the patch before use. A release liner can be applied to the topical patch material to protect the tacky patch surface before application of the patch by the user. Suitable liner materials include, but are not limited to bleached Kraft paper, silicone coated on one side at least where contact with the hydrocolloid is made. Other release liner materials include a bleached 50 gsm monolucid paper coated on one side with 20 gsm polyethylene and a silicone release formulation and having a glossy appearance on the coated side, optionally printed with removal instructions. The release liner can be of the same dimensions as the patch or can be of different dimensions to facilitate removal of the liner from the patch. Where the liner is of different dimensions from the patch, the liner can be larger in any one or all planar dimensions than the patch. Further, the liner can have lines of weakness, such as scores, perforations or a folded extension that can serve as a pull tab to facilitate removal of the liner from the patch. In one example, the release liner may have two halves separated by a wavy or other patterned full cut line that allows the user to flex the patch and easily pull back both halves of the release liner.

In one example, the modular portions may be attached by a portion of backing material or release liner, such that the modular patch portions can be removed and placed on different parts of the body. The backing material or release liner may be large enough to encompass more than one of the patch portions. The patch portions may also be separated by perforations on the backing material or release liner and the perforations may be between about 0.016 inches and about 0.281 inches, or about 0.016 inches and 0.188 inches, or about 0.016 inches and 0.094 inches.

Additionally, the topical patch, preferably a UV-cured hydrogel topical patch, may have a peel strength of between about 0 and about 325 grams-force (gf). Alternatively, the topical patch, preferably a hydrocolloid patch, may have a peel adhesion between about 9 and about 17 newtons per 25 mm. Peel adhesion is tested in accordance with ASTM D3330 (90° peel) using stainless steel or probe tack peel adhesion tests.

### Prior Art—Commercially Available Topical Patch

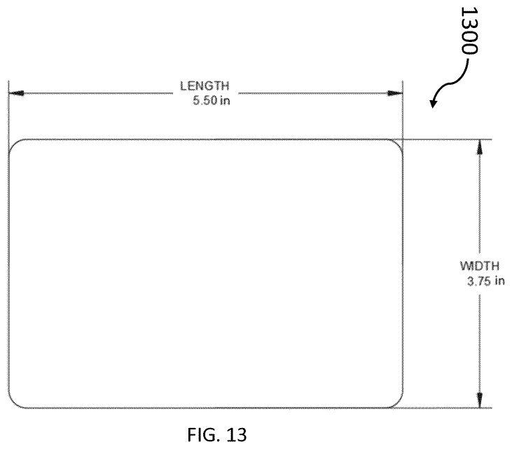

To compare against the prior art, commercially available topical patches often look like the one as shown in FIG. 13, where the patch 1300 may be approximately 5.5 inches in length and 3.75 inches in width. The mass of the commercially available rectangular patch may be about 5.83 grams. Such patches are usually advertised to be placed on large parts of the body, such as the upper or lower back. Such large patches often have adhesion challenges and start to peel or unstick during use. They may be difficult to place on smaller parts of the body and do not flex well when the body part is in motion.

In contrast to the prior art, the following examples set forth below further illustrate examples of the present invention. The present invention is not limited to the examples contained therein.

### Small Hexagon Modular Patch System

FIGS. 1A-1E depict various views of two small hexagonal topical patches 100A, 100B packaged together 110 for easy removal and modular use by a consumer. In particular, the packaging may include a backing material and/or release liner 120, such that the modular patch portions can be removed and placed on different parts of the body. The backing material may have a perforation or a full or partial cut 130 to allow a user to detach one topical patch for use, while saving the other topical patch(es) for use at a later time. The release liner 120 may include a perforation line or a full or partial cut to allow the consumer to easily open and remove the release liner to access the topical patch on the backing material. The release liner perforation line or full or partial cut may be, for example, a wavy line 140 to facilitate easy peeling and separation of the release liner from the topical patch. The wavy line 140 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

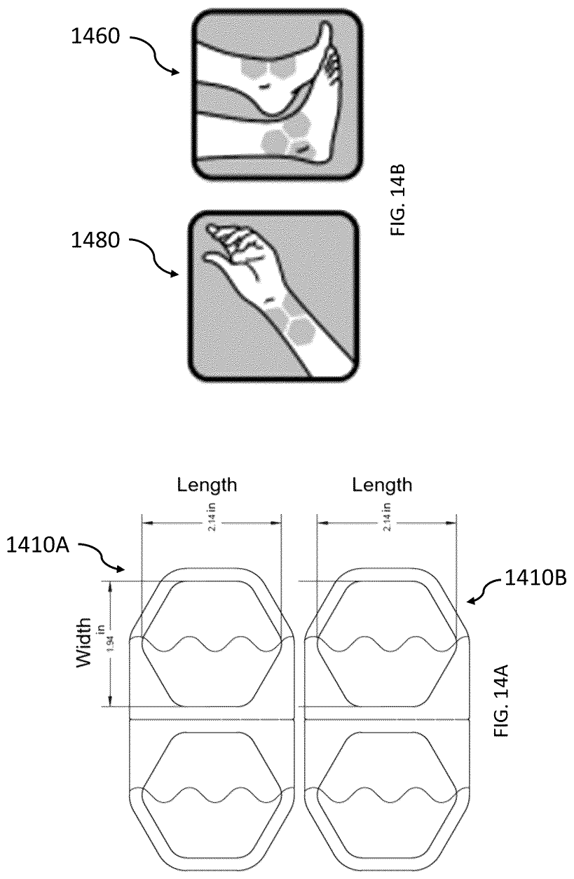

According to one example, as shown in FIG. 14B, a small hexagon patch may be used to facilitate better modular application to smaller areas of the body including the ankles 1460 and the wrist 1480. Smaller areas of the body may also include arms, feet and legs. Currently, consumers are unable to treat these areas of the body with topical patches due to the large size of commercially available patches and the lack of adhesion and flexibility of such topical patches available on the commercial market. As shown in FIG. 14A, the small hexagon patches packaged together 1410A, 1410B may be about 2.14 inches in length and about 1.94 inches in width; and about 3.65 grams in weight.

### Medium Hexagon Modular Patch System

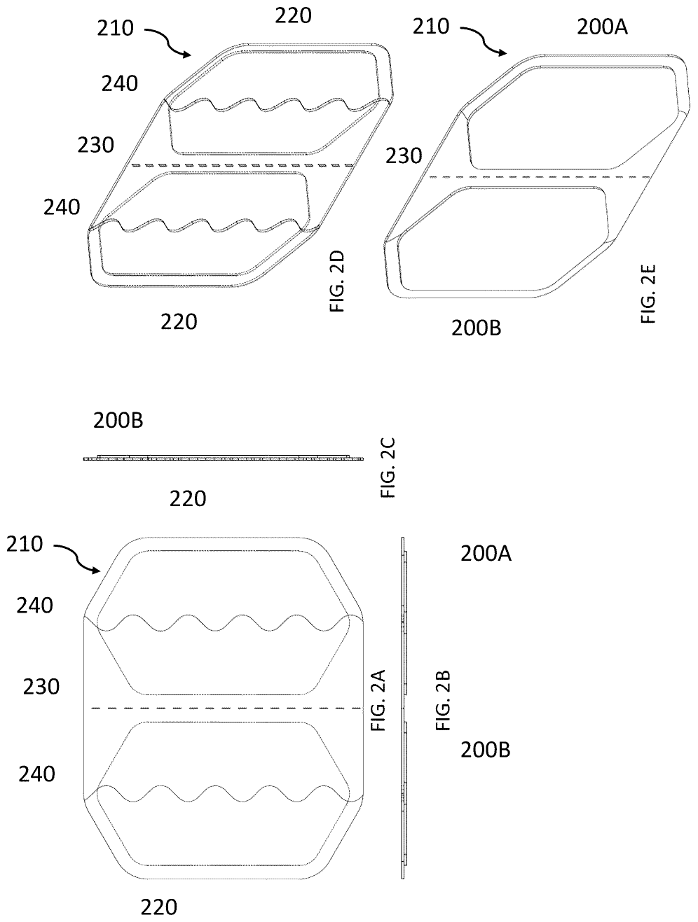

FIGS. 2A-2E depict various views of two medium hexagonal topical patches 200A, 200B packaged together 210 for easy removal and modular use by a consumer. In particular, the packaging may include a backing material and/or release liner 220, such that the modular patch portions can be removed and placed on different parts of the body. The backing material may have a perforation or a full or partial cut 230 to allow a user to detach one topical patch for use, while saving the other topical patch(es) for use at a later time. The release liner 220 may include a perforation line or a full or partial cut to allow the consumer to easily open and remove the release liner to access the topical patch on the backing material. The release liner perforation line or full or partial cut may be, for example, a wavy line 240 to facilitate easy peeling and separation of the release liner from the topical patch. The wavy line 240 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

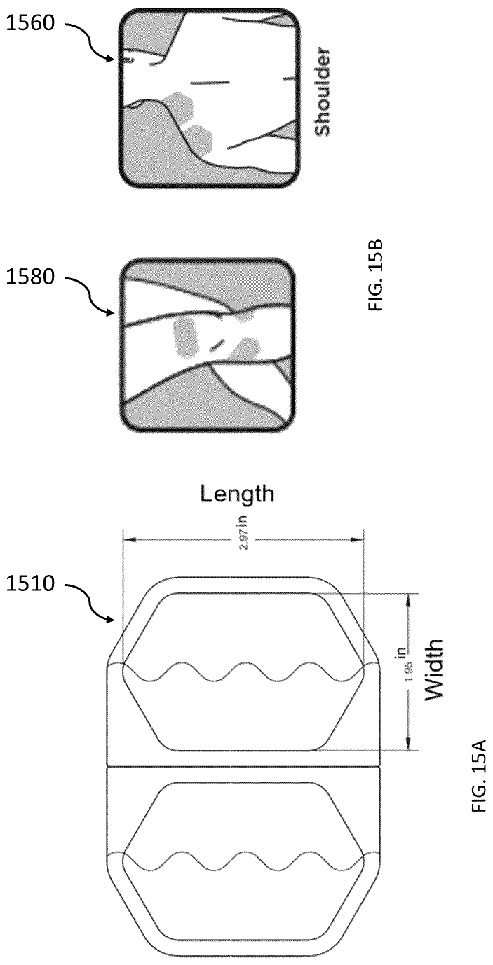

According to one example, as shown in FIG. 15B, a medium hexagon patch may be used to facilitate better modular application to smaller areas of the body, such as the shoulders 1560 and knees 1580. Smaller areas of the body may also include hips and thighs. Currently, consumers are unable to treat these areas of the body with topical patches due to the large size of commercially available patches and the lack of adhesion and flexibility of such topical patches available on the commercial market. As shown in FIG. 15A, the medium hexagon patches packaged together 1510 may be about 2.97 inches in length and about 1.95 inches in width; and about 2.74 grams in weight.

### Large Hexagon Modular Patch System

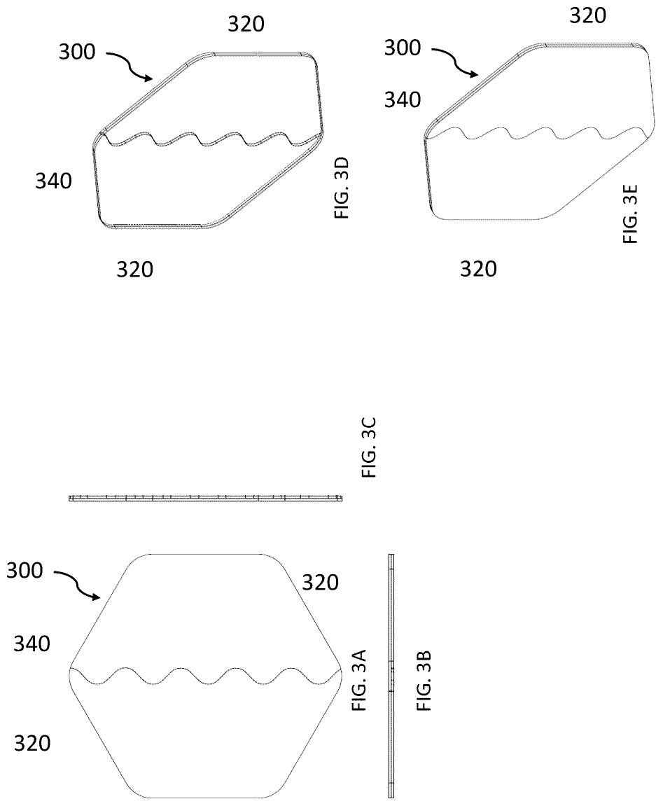

FIGS. 3A-3E depict various views of a large hexagonal topical patch 300 packaged for easy removal and modular use by a consumer. In particular, the packaging may include a backing material and/or release liner 320, such that the modular patch can be removed and placed on different parts of the body. The release liner 320 may include a perforation line or a full or partial cut to allow the consumer to easily open and remove the release liner to access the topical patch on the backing material. The release liner perforation line or full or partial cut may be, for example, a wavy line 340 to facilitate easy peeling and separation of the release liner from the topical patch. A wavy line 340 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

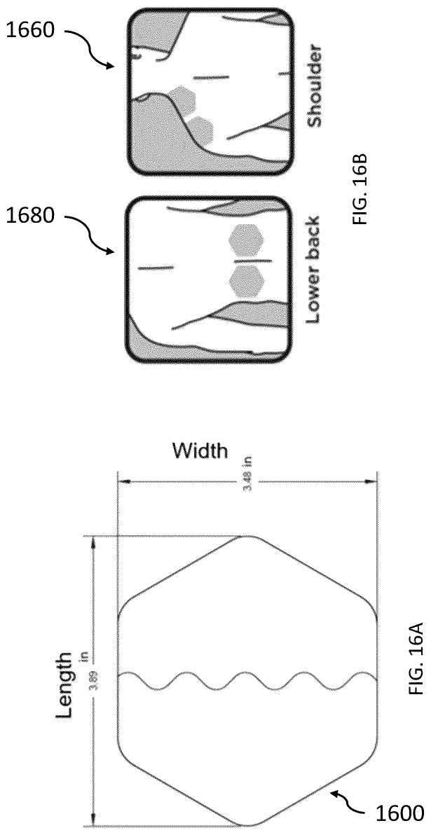

According to one example, as shown in FIG. 16B, a large hexagon patch may be used to facilitate better modular application to larger areas of the body, such as the shoulders 1660 and lower back 1680. Larger areas of the body may also include the upper back, hips and thighs. Currently, consumers are unable to effectively treat these larger areas of the body with topical patches due to the large size and lack of adhesion and flexibility of topical patches available on the commercial market. The present example offers improved adhesion and range of motion because the improved topical patch shape is less likely to bend, crease or fold, or peel or slide off. In particular, it is opined that the use of obtuse angles may preserve the structural integrity of the patch and prevent peeling. As shown in FIG. 16A, the large hexagon patch 1600 may be about 3.89 inches in length and about 3.48 inches in width; and about 5.92 grams in weight.

### Small Triangular Modular Patch System

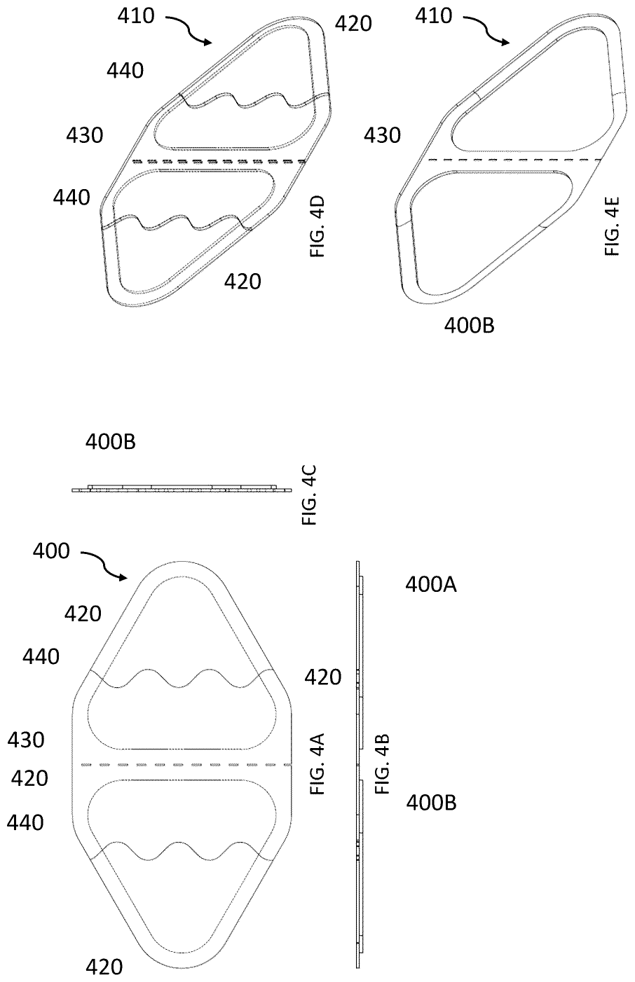

FIGS. 4A-4E depict various views of two small triangular topical patches 400A, 400B packaged together 410 for easy removal and modular use by a consumer. In particular, the packaging may include a backing material and/or release liner 420, such that the modular patch portions can be removed and placed on different parts of the body. The backing material may have a perforation or a full or partial cut 430 to allow a user to detach one topical patch for use, while saving the other topical patch(es) for use at a later time. The release liner 420 may include a perforation line or a full or partial cut to allow the consumer to easily open and remove the release liner to access the topical patch on the backing material. The release liner perforation line or full or partial cut may be, for example, a wavy line 440 to facilitate easy peeling and separation of the release liner from the topical patch. The wavy line 440 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

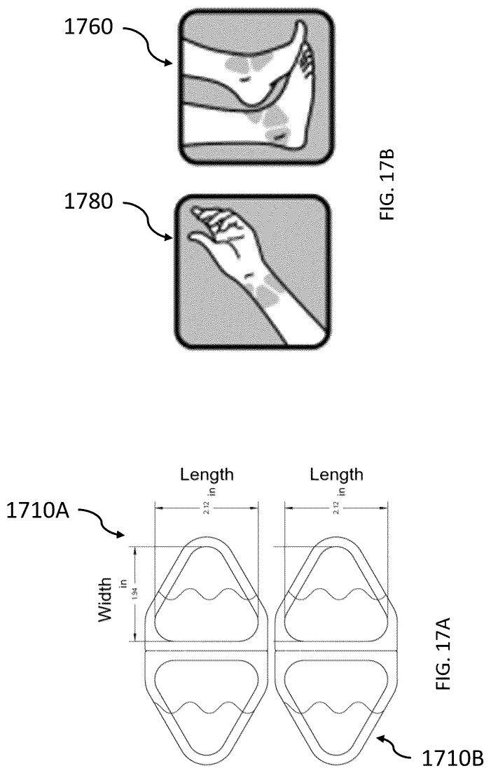

According to one example, as shown in FIG. 17B, a small triangular patch may be used to facilitate better modular application to smaller areas of the body, such as the ankles 1760 and wrist 1780. Smaller areas of the body may also include arms, feet and legs. Currently, consumers are unable to treat these areas of the body with topical patches due to the large size of commercially available patches and the lack of adhesion and flexibility of such topical patches available on the commercial market. As shown in FIG. 17A, the small triangular patches packaged together 1710A, 1710B may be about 2.12 inches in length and about 1.94 inches in width; and about 3.21 grams in weight.

### Medium Triangular Modular Patch System

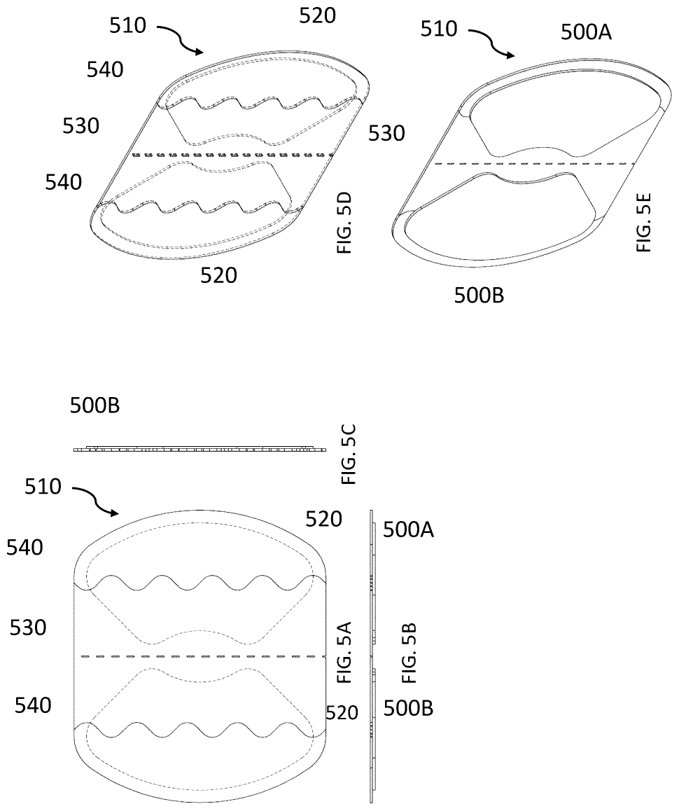

FIGS. 5A-5E depict various views of two windshield-shaped topical patches 500A, 500B packaged together 510 for easy removal and modular use by a consumer 500. In particular, the packaging may include a backing material and/or release liner 520, such that the modular patch portions can be removed and placed on different parts of the body. The backing material may have a perforation or a full or partial cut 530 to allow a user to detach one topical patch for use, while saving the other topical patch(es) for use at a later time. The release liner 520 may include a perforation line or a full or partial cut to allow the consumer to easily open and remove the release liner to access the topical patch on the backing material. The release liner perforation line or full or partial cut may be, for example, a wavy line 540 to facilitate easy peeling and separation of the release liner from the topical patch. The wavy line 540 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

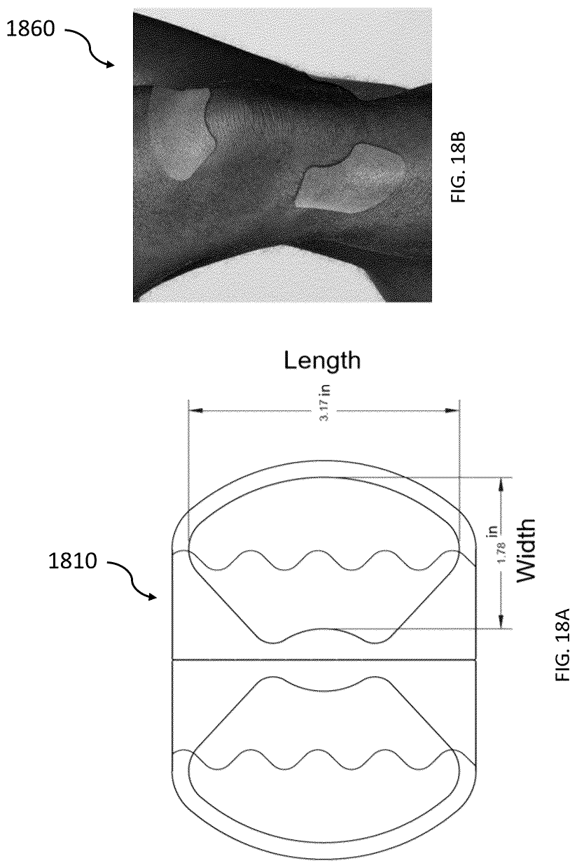

According to one example, as shown in FIG. 18B, a medium windshield-shaped patch may be used to facilitate better modular application to larger areas of the body such as the knee 1860. Larger areas of the body may also include hips and thighs. Currently, consumers are unable to treat these areas of the body with topical patches due to the large size of commercially available patches and the lack of adhesion and flexibility of such topical patches available on the commercial market.

According to an example, as shown in FIG. 18A, the medium windshield-shaped patches packaged together 1810 may optionally have curved edges and be about 3.17 inches in length and about 1.78 inches in width; and about 2.59 grams in weight. According to another example, the patch may have a corner that is cut in order to facilitate application around a joint, such as an elbow or knee. The combination of curved edges and a cut corner may be referred to as “windshield” shaped (as shown in FIG. 18A).

### Large Triangular Modular Patch System

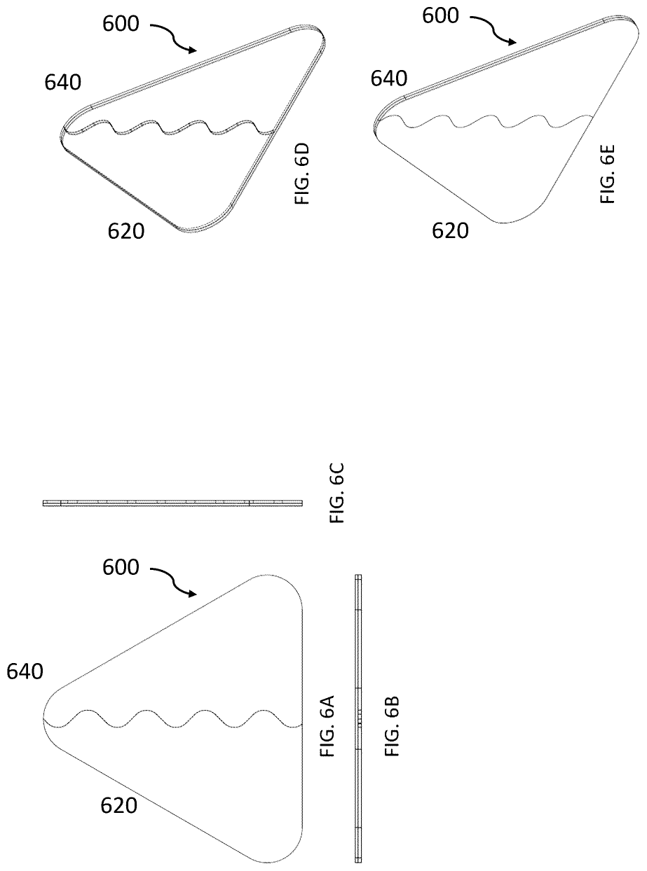

FIGS. 6A-6E depict various views of a large triangular topical patch packaged for easy removal and modular use by a consumer 600. In particular, the packaging may include a backing material and/or release liner 620, such that the modular patch can be removed and placed on different parts of the body. The release liner 620 may include a perforation line or a full or partial cut to allow the consumer to easily open and remove the release liner to access the topical patch on the backing material. The release liner perforation line or full or partial cut may be, for example, a wavy line 640 to facilitate easy peeling and separation of the release liner from the topical patch. The wavy line 640 at the center of each topical patch, in particular, allows the user to easily grab an edge or area of the release liner to peel during use. A zigzagged or other irregular line may also be envisioned.

The topical patches may be individually wrapped, or wrapped in multiples on a release liner(s), in a wrapper or pouch (free of natural rubber latex), filled into cartons, and packed out into shipping containers. Similarly sized or shaped topical patches may be packaged together in a carton or other package, or may be mixed with other topical patch sizes and shapes. Multiple wrappers or pouches of patches may be placed into the same carton.

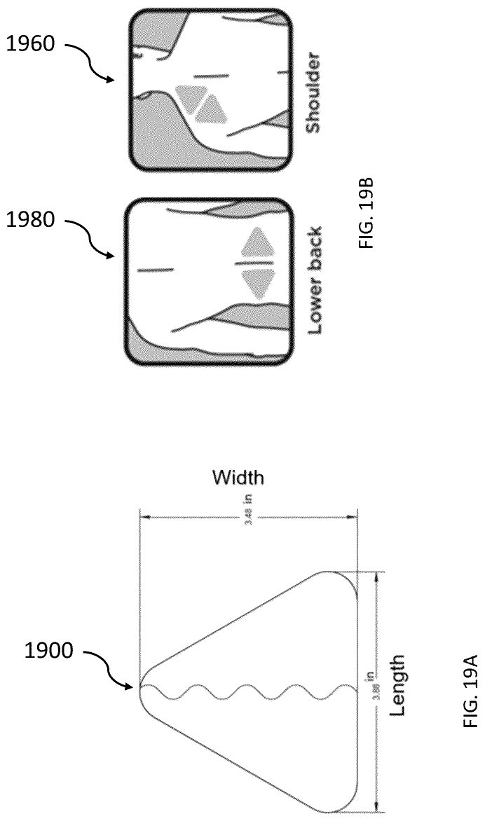

According to one example, as shown in FIG. 19B, a large triangular patch may be used to facilitate better modular application to larger areas of the body such as the shoulder 1960 and upper back 1980. Larger areas of the body may also include hips and thighs. Currently, consumers are unable to effectively treat these larger areas of the body with topical patches due to the large size and lack of adhesion and flexibility of topical patches available on the commercial market. The present example offers improved adhesion and range of motion because the improved topical patch shape is less likely to bend, crease or fold, or peel or slide off. In particular, it is opined that the use of acute angles may preserve the structural integrity of the patch and prevent peeling. As shown in FIG. 19A, the large triangular patch 1900 may be about 3.88 inches in length and about 3.48 inches in width; and about 4.86 grams in weight.

### Mixed Sized Modular Patch System

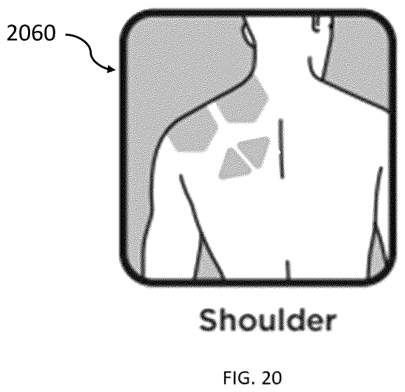

According to one example, a combination of different sized topical patches may be combined in a modular patch system. For example, the patch system may include large hexagon and/or triangular patches, medium hexagon and/or triangular patches and/or small hexagon and/or triangular patches. In the example depicted in FIG. 20, two large hexagon patches and two medium triangular patches are applied to the shoulder and upper back 2060.

### Directions for Use of Modular Patch System

The topical patches may be packaged using a backing material and/or a release liner. The patches positioned between the backing material and/or release liner may be further packaged in a sealed pouch. One or more sealed pouches may be positioned within a carton.

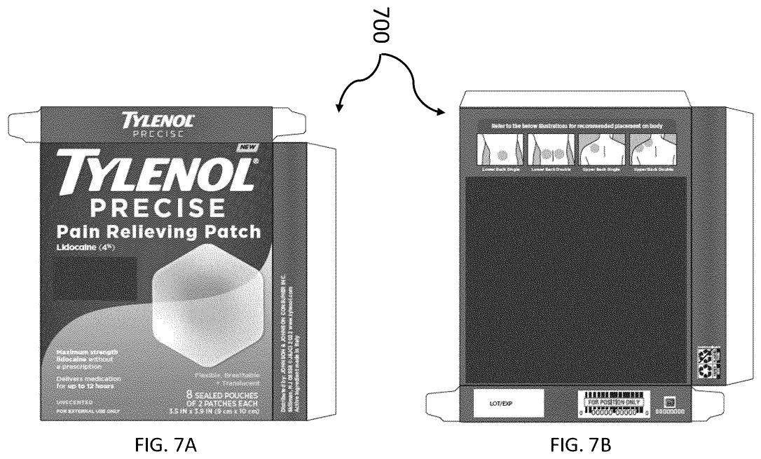

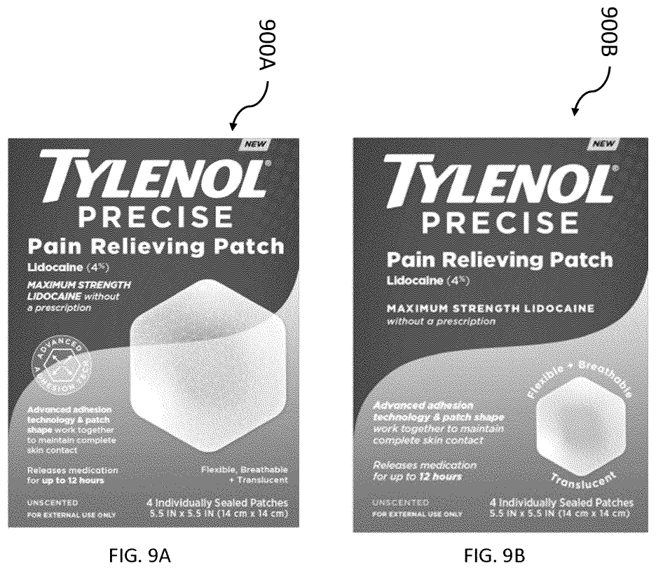

FIGS. 7A and 7B depict front and back views, respectively, of an exemplary carton 700 that may contain one or more topical patches. The carton 700 may include information (e.g., drug facts) about the topical patches, depict the shape and size of the topical patches and may include instructions for recommended placement on the body. The ornamental aspects of the carton may differentiate the topical patches from other topical patches on the market. To this end, FIGS. 9A-9B depict different ornamental alternatives 900A, 900B of the front view of the carton.

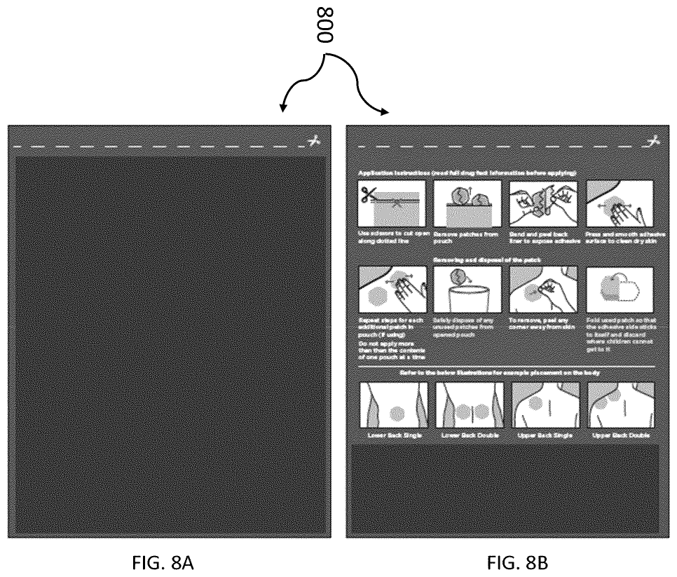

FIGS. 8A and 8B depict front and back views, respectively of an example of a pouch 800 that may contain one or more topical patches. The pouch may include information (e.g., drug facts) about the topical patches and may include application instructions to the user on how to remove the topical patches from the pouch and apply to the skin. The ornamental aspects of the pouch may differentiate the topical patches from other topical patches on the market.

In the case of a modular tropical patch system, each pouch may contain one or more topical patches that each have about 4% lidocaine or other specified amount of API.

Alternatively, in the case of a modular transdermal patch system, each pouch may contain one or more transdermal patches that collectively have no more than a specified amount of API. For example, if the transdermal patches in a pouch collectively have the specified amount of API, the pouch may include instructions not to apply more than the contents of one pouch at a time. In an alternative example, if the transdermal patches in a pouch collectively have less than the specified amount of API, the pouch may include instructions on how to combine the transdermal patches in the pouch with other topical patches so that the user only receives the maximum specified amount of API at the time of use.

According to one example, a user is directed to the following application instructions for removal of the patches from the pouch and application to the skin:
Use scissors to cut (the pouch) open along the dotted line.
Remove patches from the pouch.
Bend and peel back the liner to expose adhesive.
Press and smooth adhesive surface (of patch) to clean dry skin.
Repeat steps for each additional patch in pouch (if using).
(Optionally) Do not apply more than the contents of one pouch at a time.
Safely dispose of any unused patches from opened patch.

In one alternative example, the pouch may be resealable allowing unused patches to be saved for later use rather than disposed of.

According to an example, the application instructions include further directions for removal of the patch from the user's skin:
To remove, peel any corner (of the patch) away from the skin.
Fold used patch so that the adhesive side sticks to itself and discard where children cannot get to it.

### Human Use Testing

Various aspects of the exemplary modular topical patch system may be tested with consumers to verify enhanced efficacy and ease of use over topical patches currently available on the market. Testing criteria may include: 1) advanced patch technology that stays in place, 2) stays in place, 3) sweat-resistant, 4) flexible, 5) ultra-flexible, 6) breathable, 7) transparent/translucent design, 8) translucent, 9) transparent, 10) made with fully breathable, transparent materials, 11) comfortably fits like a second skin, 12) releases medication all day for relief, or up to a certain number of hours, 13) unscented/fragrance free, 14) no residue, 15) designed to move with you, 16) moves with you, 17) patch shape contours to your skin, 18) patch shape resists pulling or loosening no matter how you move, 19) advanced adhesion technology & patch shape work together to maintain complete skin contact, even in high flex areas, 20) maintains complete skin contact, even in high flex areas, 21) move naturally with your pain without your patch holding you back, 22) allows me to move naturally, 23) ideal for discreet, comfortable wear anywhere on your body, 24) is discreet, 25) is comfortable, and/or 26) fits like second skin.

### Alternative Application

In addition to the modular patch system described above for pain relief, other applications may be further envisioned. For example, the modular patch system may be envisioned for use with consumers having sensitive skin to treat various skin health conditions, such as acne, contact dermatitis, dry skin, itchy skin and eczema.

In one example, the modular patch system may deliver a plant-based ingredient or extract to the skin; examples may include Boswellia, arnica, resveratrol, aloe vera and/or oat. Alternatively, the modular patch system may deliver a steroid ingredient, for example an anti-inflammatory ingredient for eczema.

In one example, the modular patch system may deliver a processed oat ingredient to the skin, which has been shown to be effective in treating sensitive skin, including skin that is dry, rough, flaky and itchy. A “processed oat ingredient” refers to an ingredient that is typically derived from a part of the oat plant (Avena sativa). Processed oat ingredients are known to have antioxidant, antifungal and anti-inflammatory properties. The processed oat ingredient can be either a processing (e.g., an extract, milling, fermenting) of one or more parts of the oat plant (e.g., grain, leaf, stem, seed) or can be a molecule found in the oat plant (e.g., beta-glucan, flavonoids, avenanthramides, lipids, peptides, etc.). This definition is intended to cover processed oat ingredients which are derived from other sources other than oat (e.g., from another plant or chemically synthesized), but are otherwise associated with oat. In one or more examples, the processed oat ingredient is selected from the group consisting of oat extract, colloidal oatmeal, colloidal oat flour, oat flour, oat bran, oat protein, oat peptide, oat oil, fermented oat, avenanthramides, beta-glucan, modified oat grain material (e.g., chemically, enzymatically-, microorganism-modified), and combinations thereof.

As used herein, “colloidal oat flour” or “colloidal oatmeal” means the powder resulting from the grinding and further processing of whole oat grain meeting United States Standards for Number 1 or Number 2 oats. The colloidal oat flour has a particle size distribution as follows: not more than 3 percent of the total particles exceed 150 micrometers in size and not more than 20 percent of the total particles exceed 75 micrometers in size. Examples of suitable colloidal oat flours include, but are not limited to, “Tech-0” available from the Beacon Corporation and colloidal oat flour available from Quaker.

The processed oat ingredient(s) may be present in amounts ranging from about 0.01, 0.05, 0.1, 0.2, 0.3, 0.4, 0.5, 0.6, 0.7, 0.8, 0.9, 1.0, 1.1, 1.2, 1.3, 1.4, 1.5, 2, 2.5, 3, 3.5, 4, 4.5, or 5 to about 0.5, 0.6, 0.7, 0.8, 0.9, 1, 1.1, 1.2, 1.3, 1.4, 1.5, 1.6, 1.7, 1.8, 1.9, 2, 2.5, 3, 3.5, 4, 5, 6, 7, 8, 9, 10 wt. % of the total composition. In one or more examples, the processed oat ingredient(s) is present in an amount ranging from about 0.1 wt. % to about 10 wt. % by total weight of the composition. In some examples, the processed oat ingredient(s) is present in an amount ranging from about 0.1 wt. % to about 5 wt. % by total weight of the composition.

In another example, the patch system may deliver a “humectant” defined as a compound intended to increase the water content of the top layers of skin (e.g., hygroscopic compounds). Examples of suitable humectants include, but are not limited to, glycerin, sorbitol or trehalose (e.g., α,α-trehalose, β,β-trehalose, α,β-trehalose) or a salt or ester thereof (e.g., trehalose 6-phosphate). In one example, the humectant is glycerin (also spelled “glycerine” interchangeably).

In another example, the modular patch system may deliver a processed oat ingredient and a humectant, for example, glycerin. In one example, the processed oat ingredient may be a combination of oat ingredients, e.g., colloidal oat, oat flour and/or oat extract.

A modular patch system would benefit a skin health application by allowing a user to reach and affix topical patches to smaller portions of the body, such as under the eye, elbows, hands, wrists, fingers and other portions of the body where users may have sensitive skin. The discreetness of the modular patch system is also beneficial where a user is applying the patch to visible areas of the body, such as the face, arms, wrists and hands.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A modular topical patch system, comprising two or more topical patches that share a commonality to allow them to be affixed adjacent, parallel or in close proximity to one another on skin of a user to facilitate pain relief.

2. The modular topical patch system of claim 1, wherein the commonality is a common dimension, a common angle, a complementary angle, a common shape and/or a complementary shape.

3. The modular topical patch system of claim 2, wherein the common angle is an obtuse angle or an acute angle.

4. The modular topical patch system of claim 2, wherein the complementary angle of a first patch is an obtuse angle and the complementary angle of a second patch is an acute angle.

5. The modular topical patch system of claim 1, wherein the two or more topical patches are positioned on the skin at a smaller joint or body location of the user.

6. A method of relieving pain, comprising a user affixing two or more topical patches adjacent, parallel or in close proximity to one another on skin of a user to facilitate pain relief, wherein the two or more topical patches share a commonality that facilitates modular application to the skin.

7. The method of claim 6, wherein the commonality is a common dimension, a common angle, a complementary angle, a common shape and/or a complementary shape.

8. The method of claim 7, wherein the common angle is an obtuse angle or an acute angle.

9. The method of claim 7, wherein the complementary angle of a first patch is an obtuse angle and the complementary angle of a second patch is an acute angle.

10. The method of claim 6, wherein the two or more topical patches are positioned on the skin at a smaller joint or body location of the user.

11. We further claim the ornamental design for a modular topical patch system as shown and described in FIGS. 1A through 9B.
